(19) United States
(12) Patent Application Publication
(10) Pub. No.: US 2025/0009793 A1
(43) Pub. Date: Jan. 9, 2025

(54) METHODS AND COMPOSITIONS FOR REPAIR OF TENDON-BONE INTERFACE

(71) Applicant: MAYO FOUNDATION FOR MEDICAL EDUCATION AND RESEARCH, Rochester, MN (US)

(72) Inventors: Atta Behfar, Rochester, MN (US); Chunfeng D. Zhao, Rochester, MN (US)

(21) Appl. No.: 18/709,931

(22) PCT Filed: Nov. 16, 2022
(86) PCT No.: PCT/US2022/050125

§ 371 Date: May 14, 2024

Related U.S. Application Data
(60) Provisional application No. 63/279,841, filed on Nov. 16, 2021.

(51) Int. Cl. A61K 35/12; A61K 9/00; A61P 19/04

(52) U.S. Cl. CPC A61K 35/12; A61K 9/0012; A61P 19/04

(57) ABSTRACT

A method of repairing a damaged bone-tendon interface in a subject generally includes contacting the damaged bone-tendon interface with an effective amount of a composition that includes purified exosome product (PEP) and a pharmaceutically acceptable carrier. In one or more embodiments, the damaged bone-tendon interface includes complete separation of tendon from bone and the method further includes surgically reattaching the tendon to the bone. In one or more embodiments, the damaged tendon-bone interface comprises partial separation of tendon from bone and the method includes implanting the PEP composition at a site effective for contacting the PEP composition with the damaged tendon-bone interface.

### CROSS-REFERENCE TO RELATED APPLICATIONS

This application claims the benefit of U.S. Provisional Application Ser. No. 63/279,841, filed on Nov. 16, 2021, which is incorporated by reference herein in its entirety.

### SUMMARY

This disclosure describes, in one aspect, a method of repairing a damaged bone-tendon interface in a subject. Generally, the method includes contacting the damaged bone-tendon interface with an effective amount of a composition that includes purified exosome product (PEP) and a pharmaceutically acceptable carrier.

In one or more embodiments, the PEP includes spherical or spheroid exosomes having a diameter no greater than 300 nm.

In one or more embodiments, the PEP includes spherical or spheroid exosomes having a diameter of from 56 nm to 151 nm.

In one or more embodiments, the PEP includes spherical or spheroid exosomes having a mean diameter of 97 nm. In one or more of these embodiments, the PEP includes spherical or spheroid exosomes having a mean diameter of 97 nm±54 nm.

In one or more embodiments, the PEP includes from 1% to 20% CD63⁻ exosomes and from 80% to 99% CD63⁺ exosomes.

In one or more embodiments the PEP includes at least 50% CD63⁻ exosomes.

In one or more embodiments, the PEP includes from 1×10¹¹ PEP exosomes to 1×10¹³ PEP exosomes.

In one or more embodiments, the PEP includes from 1×10¹² PEP exosomes to 1×10¹³ PEP exosomes.

In one or more embodiments, the composition further includes a supportive matrix. In one or more of these embodiments, the supportive matrix includes a collagen scaffold. The supportive matrix may additionally include a tissue sealant or a fibrin sealant.

In one or more embodiments, an effective amount is an amount effective to increase osteoblast-tenocyte interface compared to osteoblast-tenocyte interface of a bone-tendon interface treated without PEP.

In one or more embodiments, an effective amount is an amount effective to improve at least one histological measure of the tendon-bone interface compared to a bone-tendon interface treated without PEP.

In one or more embodiments, the histological measure includes an increase fiber continuity, an increase fiber parallel orientation, an increase collagen fiber density, a decrease vascularity, or a decrease cellularity compared to a bone-tendon interface treated without PEP.

In one or more embodiments, an effective amount is an amount effective to increase expression of at least one gene that promotes repair of a damaged tendon-bone interface. In one or more of these embodiments, the gene encodes type I fibrillar collagen (Col1), type III fibrillar collagen (Col3), scleraxis BHLH transcription factor (SCX), tenomodulin (TNMD), decorin (DCN), or insulin-like growth factor 1 (IGF-I) in tissues of the tendon-bone interface. In one or more embodiment, an effective amount is an amount effective to increase at least one biomechanical measure of the tendon-bone interface compared to a bone-tendon interface treated without PEP. The biomechanical measure may include, for example, maximum load or stiffness.

In one or more embodiments, the damaged bone-tendon interface includes complete separation of tendon from bone; and the method further includes surgically reattaching the tendon to the bone.

In one or more embodiments, the damaged tendon-bone interface includes partial separation of tendon from bone; and the method includes implanting the PEP composition at a site effective for contacting the PEP composition with the damaged tendon-bone interface.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Co-culture model and cell identification. (A) Schematic representation of the co-culture model, including osteoblasts, tenocytes, and PEP gel cube. (B) The boundary was cut and removed when cells reached the boundary. (C) Schematic representation of the osteoblast region, tenocyte region, and interface region in the co-culture model. (D) Photograph of the co-culture model before the boundary was removed. (E) Photograph of the co-culture model after the boundary was removed. (F) Alkaline phosphatase staining after application of normal culture medium, with PEP, or osteogenic induction conditions, day 7 and day 14. (G) Relative mRNA expression levels of Col1, Col3, and SCX in primary tenocytes (osteoblasts as the control group). Labels: ALP, alkaline phosphatase; PEP, purified exosome product; *, P<0.1; **, P<0.01; * P<0.001.

FIG. 2. Morphologic characterization of PEP. (A) PEP was formulated and stored in a stabilized lyophilized powder form in vials to allow for room temperature storage. (B) Preparation of fibrin sealant (TISSEEL, Baxter International, Inc., Deerfield, IL) with and without PEP. A vial of sealed PEP powder was mixed with 1 mL phosphate-buffered saline (PBS) to prepare the 100% (vol/vol) PEP solution. PEP solution (400 μL) was added to the 600-μL CaCl₂) solution, and the solution was normalized to a 40% (vol/vol) concentration. Manufacturer directions for TISSEEL preparation were then used to finish preparation. The final concentration of PEP in TISSEEL was 20% (vol/vol). (C) PEP has a spherical vesicle structure with intact lipid-bilayer. (D) Particle size distribution analysis (NanoSight Ltd., Salisbury, United Kingdom) showing that average vesicle diameter of PEP ranged from 56.4 nm to 151 nm and the mean diameter was 96.9 nm+2.8 nm, representing the standard size range of exosomes. The 100% PEP solution was calculated to be 1.9×10¹¹ particles/mL.

FIG. 3. Flowchart showing the experimental design in the in vivo model. The same rats were used in each evaluation marked with “*”. Labels: PEP, purified exosome product; H&E, hematoxylin-eosin; RC, rotator cuff.

FIG. 4. Surgical procedure and biomechanical testing of tendon-bone interface repair in a rotator cuff model. (A) Local PEP placement at insertion site of the supraspinatus tendon. (B) Modified Mason-Allen suture. (C) Gross observation of PEP gel cube before implantation in vivo. (D) PEP gel cube placed before suturing. (E) Two double-armed 5-0 sutures (ETHIBOND, Ethicon Inc., Raritan, NJ) were passed through the tendon transversely, and small loops were made on both sides of the tendon. (F) The supraspinatus tendon was transected at its insertion site on the greater tuberosity. (G) Suture was passed through a 0.5-mm hole drilled transversely at the proximal part of the humerus. (H) Gross observation after careful suturing. (I) The biomechanical testing system shows the humerus embedded within a tube of polymethylmethacrylate. The supraspinatus tendon is fixed to the attachment through a clamp at ultimate load to failure. (J) Maximum load to failure at six weeks post-surgery was significantly higher in the TISSEEL-PEP group compared to the repair-only group. (K) Stiffness at six weeks post-surgery was significantly higher in the TISSEEL-PEP group compared to the repair-only group. Results in (J) and (K) are shown as mean (SD) (n=8 for each group). Labels: PEP, purified exosome product; RC, rotator cuff, *, P<0.1; ***, P<0.001; ****, P<0.0001.

FIG. 5. Cell growth and migration following exposure to PEP. Blue indicates the region of osteoblasts, and red indicates the region of tenocytes at various time points (0 days, 2 days, 4 days, 6 days, and 8 days, n=6). Left, PEP group. Right, control group (scale bars, 200 m). Histograms show quantified results of the gap area and the fusion area.

FIG. 6. Result of quantitative RT-PCR verification in the in vitro trial. (A) Real-time PCR results of Col1 mRNA expression three days after direct contact of osteoblasts and tenocytes in the in vitro trial. (B) RT-PCR results of Col3 mRNA expression three days after direct contact of osteoblasts and tenocytes in the in vitro trial. (C) RT-PCR results of DCN mRNA expression three days after direct contact of osteoblasts and tenocytes in the in vitro trial. (D) RT-PCR results of TNC mRNA expression three days after direct contact of osteoblasts and tenocytes in the in vitro trial. (E) RT-PCR results of Spp1 mRNA expression three days after direct contact of osteoblasts and tenocytes in the in vitro trial. (F) RT-PCR results of EGR mRNA expression three days after direct contact of osteoblasts and tenocytes in the in vitro trial. (G) RT-PCR results of PPARG mRNA expression three days after direct contact of osteoblasts and tenocytes in the in vitro trial. RT-PCR, reverse transcription-polymerase chain reaction; *, P<0.1; ** P<0.01; ***, P<0.001.

FIG. 7. Histologic analysis and result of quantitative RT-PCR verification in the in vivo trial. Histology of rat rotator cuff tendon and its insertion into the humerus after six weeks. (A) Normal control group. (B) Repair-alone group. (C) TISSEEL alone group. (D) TISSEEL-PEP group. Histological staining for each of (A)-(D): (1) H&E staining (×10 magnification), (2) Masson trichrome staining (×10 magnification, black frame, ×20 magnification), (3) Picrosirius red stain (×10 magnification), and (4) Picrosirius red stain under a polarizing microscope; scale=100 μM.). (E) Histologic findings were evaluated using a semiquantitative scoring system. (F) Real-time PCR results of relative mRNA expression of Col1, Co3, SCX, Tnmd, TNC, DCN, Spp1, and IGF-I after six weeks in the in vivo trial. H&E, hematoxylin-eosin; PEP, purified exosome product; RC, rotator cuff, RT-PCR, reverse transcription-polymerase chain reaction; *, P<0.1; ** P<0.01; ***, P<0.001; ****, P<0.0001.

### DETAILED DESCRIPTION OF ILLUSTRATIVE EMBODIMENTS

This disclosure describes compositions and methods that promote healing and/or repair of damage at the tendon-bone interface. While described herein in the context of an exemplary model of tendon-bone interface involving repairing a rotator cuff tear, the methods described herein may be practiced to repair and/or heal a damaged tendon-bone interface having a lesser degree of damage. Moreover, the methods described herein may be practiced to repair and/or heal any damaged tendon-bone interface at any site in the body.

This disclosure describes the effects of a purified exosome product (PEP) on osteoblasts and tenocytes in a new co-culture model, using PEP to improve tendon-bone healing in a rat rotator cuff tear model, and the molecular mechanism by which PEP induces tendon-bone healing. PEP upregulates tendon-bone interface healing by promoting tenocyte proliferation and migration. Thus, treatment of the tendon-bone interface—e.g., by local implantation of PEP increases expression of genes and/or signal pathways that promote enthesis healing.

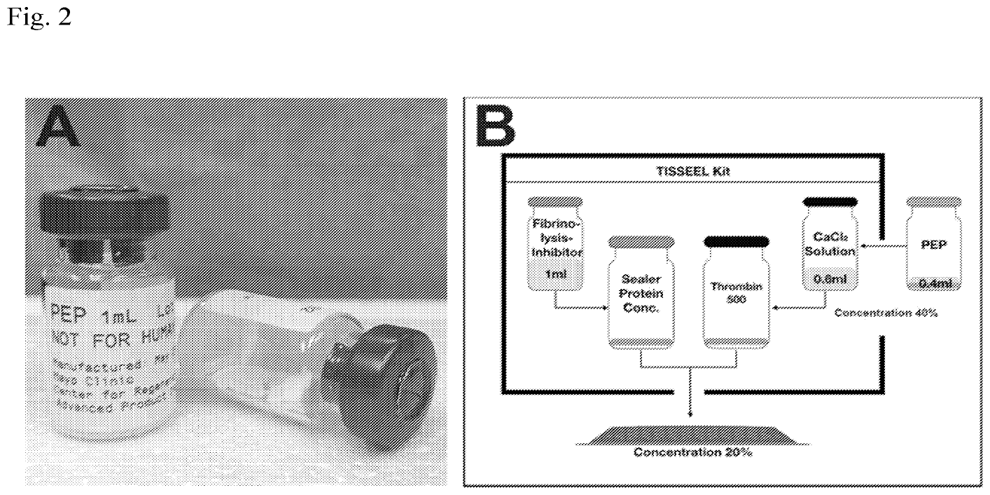

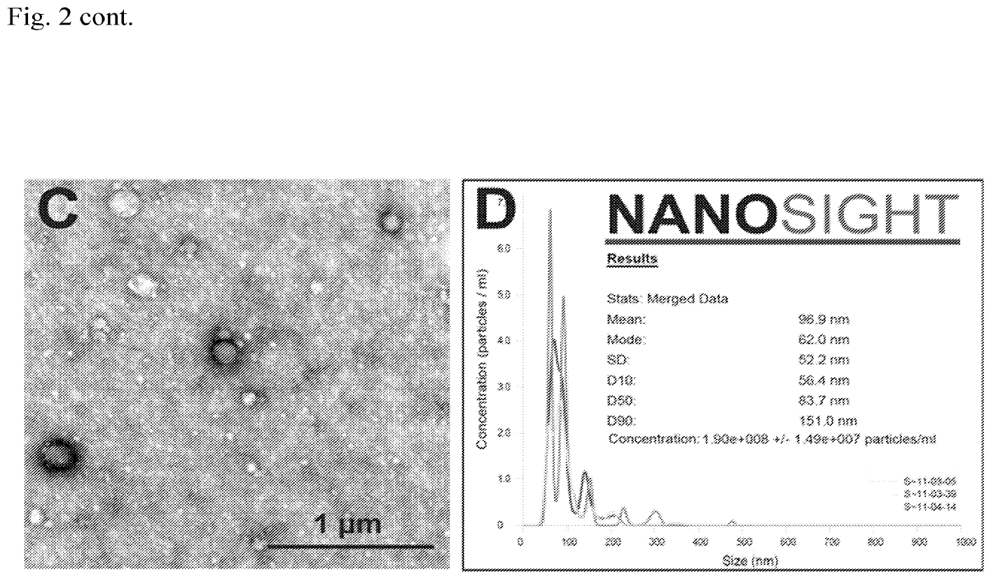

PEP is a purified exosome product prepared using a cryodesiccation step that produces a product having a structure that is distinct from exosomes prepared using conventional methods. For example, PEP typically has a spherical or spheroidal structure and an intact lipid bilayer rather than a crystalline structure that results from the reaggregation of lipids of the exosome lipid bilayer after exosomes are disrupted during convention exosome preparation methods. As used herein, a “spheroid” structure is shaped like a three-dimensional sphere with flattened poles. The spherical or spheroid exosome structures generally have a diameter of no more than 300 nanometers (nm). Typically, a PEP preparation contains spherical or spheroid exosome structures that have a relatively narrow size distribution. An example size distribution of a PEP preparation is shown in FIG. 2D. Here, the mean particle size diameter was 96.9 nm±52.2 nm. In some preparations, PEP includes spherical or spheroidal exosome structures with a mean diameter of 110 nm±90 nm, with most of the exosome structures having a mean diameter of 110 nm±50 nm such as, for example, 110 nm±30 nm.

An unmodified PEP preparation—i.e., a PEP preparation whose character is unchanged by sorting or segregating populations of exosomes in the preparation-naturally includes a mixture of CD63⁺ and CD63⁻ exosomes. Because CD63⁻ exosomes can inhibit unrestrained cell growth, an unmodified PEP preparation that naturally includes CD63⁺ and CD63⁻ exosomes can both stimulate cell growth for wound repair and/or tissue regeneration and limit unrestrained cell growth.

Further, by sorting CD63⁺ exosomes, one can control the ratio of CD63⁺ exosomes to CD63⁻ exosomes in a PEP product by removing CD63⁺ exosomes from the naturally-isolated PEP preparation, then adding back a desired amount of CD63⁺ exosomes. In one or more embodiments, a PEP preparation can have only CD63⁻ exosomes.

In one or more embodiments, a PEP preparation can have both CD63⁺ exosomes and CD63⁻ exosomes. The ratio of CD63⁺ exosomes to CD63⁻ exosomes can vary depending, at least in part, on the quantity of cell growth desired in a particular application. For example, a CD63⁺/CD63⁻ exosome ratio provides desired cell growth induced by the CD63⁺ exosomes and inhibition of cell growth provided by the CD63⁻ exosomes achieved via cell-contact inhibition. In certain scenarios, such as in tissues where non-adherent cells exist (e.g., blood derived components), this ratio may be adjusted to provide an appropriate balance of cell growth or cell inhibition for the tissue being treated. Since cell-to-cell contact is not a cue in, for example, tissue with non-adherent cells, one may reduce the CD63⁺ exosome ratio to avoid uncontrolled cell growth. Conversely, if there is a desire to expand out a clonal population of cells, such as in allogeneic cell-based therapy or immunotherapy, one can increase the ratio of CD63⁺ exosomes to ensure that a large population of cells can be derived from a very small source.

Thus, in one or more embodiments, the ratio of CD63⁺ exosomes to CD63⁻ exosomes in a PEP preparation may be at least 1:1, at least 2:1, at least 3:1, at least 4:1, at least 5:1, at least 6:1, at least 7:1, at least 8:1, at least 9:1, at least 10:1, at least 11:1, at least 12:1, at least 13:1, at least 14:1, at least 15:1, or at least 16:1. In one or more embodiments, the ratio of CD63⁺ exosomes to CD63⁻ exosomes in a PEP preparation may be at most 15:1, at most 16:1, at most 17:1, at most 18:1, at most 19:1, at most 20:1, at most 25:1, or at most 30:1. For example, the ratio of CD63⁺ exosomes to CD63⁻ exosomes may be between 1:1 to 30:1, 2:1 to 20:1, 4:1 to 15:1, or 8:1 to 10:1. In one or more certain embodiments, the PEP product is formulated to contain a 9:1 ratio of CD63⁺ exosomes to CD63⁻ exosomes. In one or more certain embodiments, native PEP, e.g., PEP with an unmodified ratio of CD63⁺ exosomes to CD63⁻ exosomes may be used.

Production of purified exosome product (PEP) involves separating plasma from blood and isolating a solution of exosomes from separated plasma with filtration and centrifugation. PEP is fully characterized and methods for preparing PEP are described in International Patent Application No. PCT/US2018/065627 (published as International Publication No. WO 2019/118817), U.S. Patent Publication No. 2021/0169812 A1, and U.S. Pat. No. 10,596,123, each of which is incorporated by reference herein in its entirety.

### Morphologic Characterization of PEP

In one or more embodiments, the vesicle size of PEP exosomes may be measured to characterize the preparation. Vesicle size may be measured, for example, by electron microscopy, such as transmission electron microscopy or scanning electron microscopy. Transmission electron microscopy images indicate that exosomes of PEP exhibit the typical spherical vesicles with an intact lipid-bilayer structure (FIG. 2C). Vesicle sizes of PEP ranged from 56.4 nm to 151 nm with a mean size of 96.9 nm+2.8 nm, representing the standard size range of exosomes. PEP solution at 100% was calculated to contain 1.9×10¹¹ PEP particles/mL (FIG. 2D).

### In Vitro Experiments

As described herein, an in vitro cell culture model of tendon-bone interface repair may be prepared using primary osteoblasts and tenocytes. This culture model may be used to determine the effect of PEP or another pharmaceutical composition on tendon-bone interface repair.

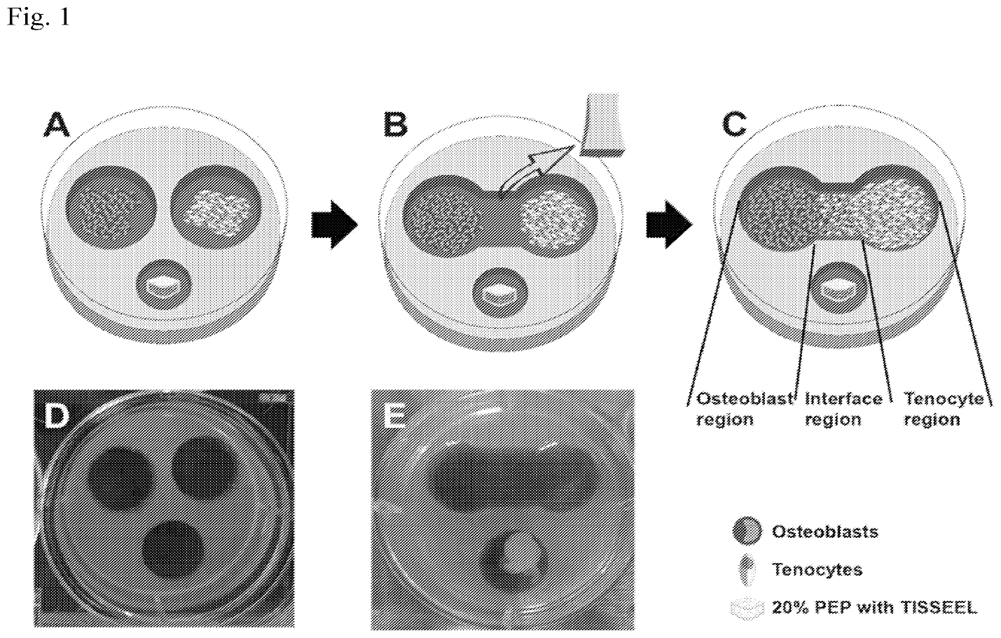

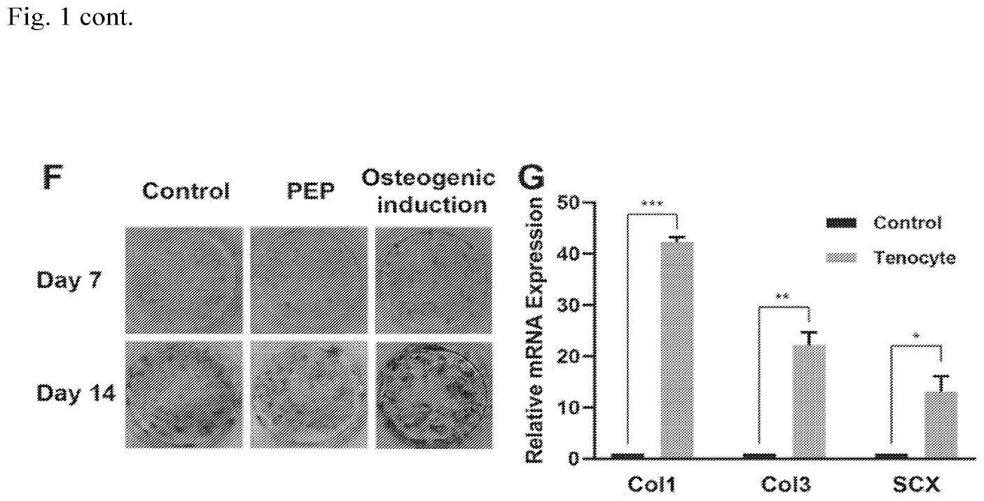

Identification of Rat Primary Osteoblasts and Tenocytes Primary cells from any organism of interest, such as mouse, rat, horse, dog, pig, or primate, may be used. Calvaria cells isolated from neonatal rats were selected as rat primary osteoblasts for in vitro studies. The alkaline phosphatase (ALP) activity of osteoblasts was gradually increased as the cultivation time prolonged (from day 7 to day 14). In osteogenic induction media, the osteoblasts showed more osteogenesis and enhanced further as the culture time was increased (FIG. 1F). However, the PEP group did not show more osteogenic capacity. This result suggests that the 20% TISSEEL-PEP did not enhance osteoblast activity when compared to control under osteogenic conditions in an vitro co-culture model.

PCR results showed that the relative mRNA expression of tendon-related genes was significantly upregulated compared with the control (osteoblast) group. Furthermore, SCX was directly related to tendon development and differentiation, indicating that the identification of the primary cell type corresponds very well to the tendon-origin (FIG. 1G).

### Effects of PEP on Morphology and Outgrowth of the Interface Region

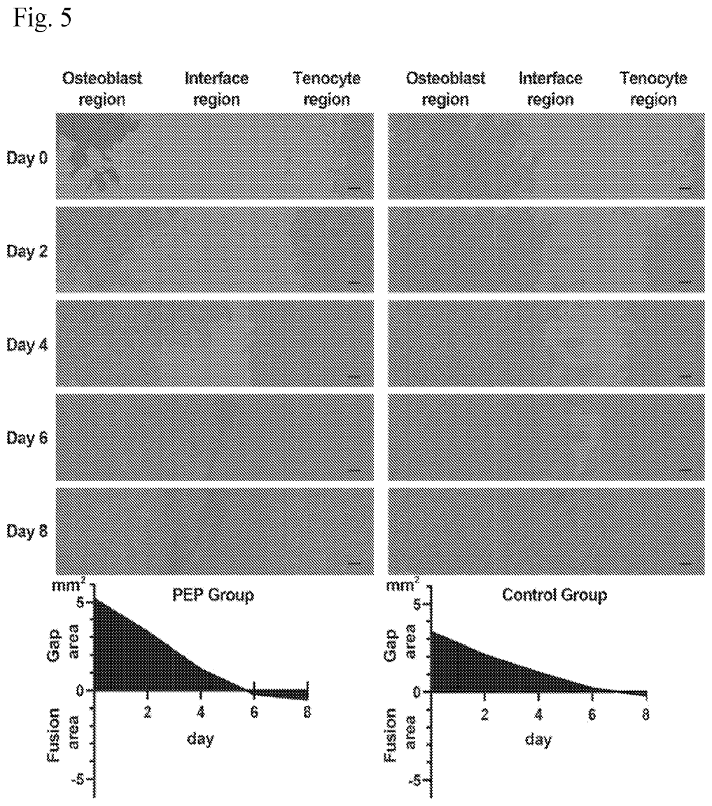

The in vitro culture model illustrated in FIG. 1A-E allowed evaluation of the migration and fusion of osteoblasts and tenocytes with and without PEP treatment. Histologic growth patterns of the osteoblast region, tenocyte region, and interface region were manually pseudocolored at days 0, 2, 4, 6, and 8 (FIG. 5). Area coverages were manually measured to calculate gap area and fusion area. The initial gap area of the intervention group (PEP group) was 5.17±0.22 mm² (n=6), while the initial gap area of the control group (no PEP) was 3.37±0.19 mm² (n=6). Cell growth was significantly increased in all regions following exposure to PEP. Cell migration was more significant in the interface of the PEP group compared to the control group. In the PEP group, cells had been confluent at day 4, and the fusion area was 0.47±0.14 mm² (n=6) at day 8. In the control group, however, cells did not begin to become confluent until day 6, and the fusion area was only 0.20±0.17 mm² (n=6) at day 8 (FIG. 5).

Changes of mRNA Levels

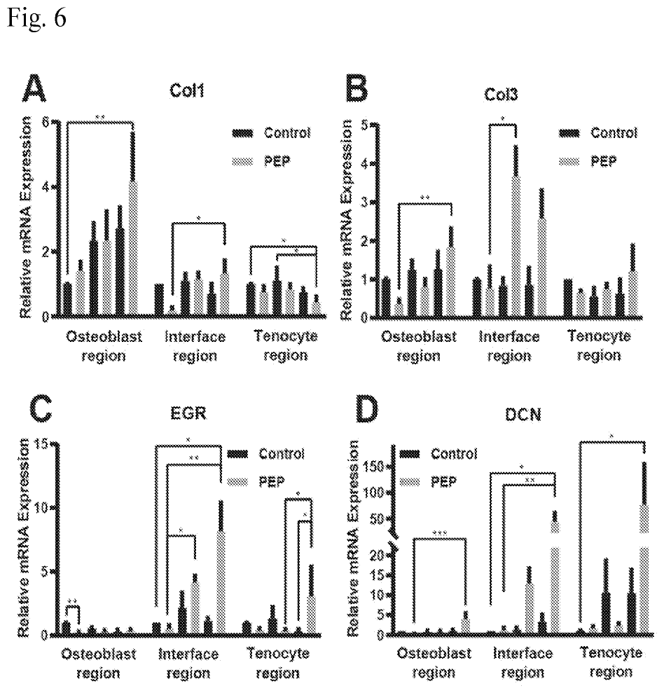

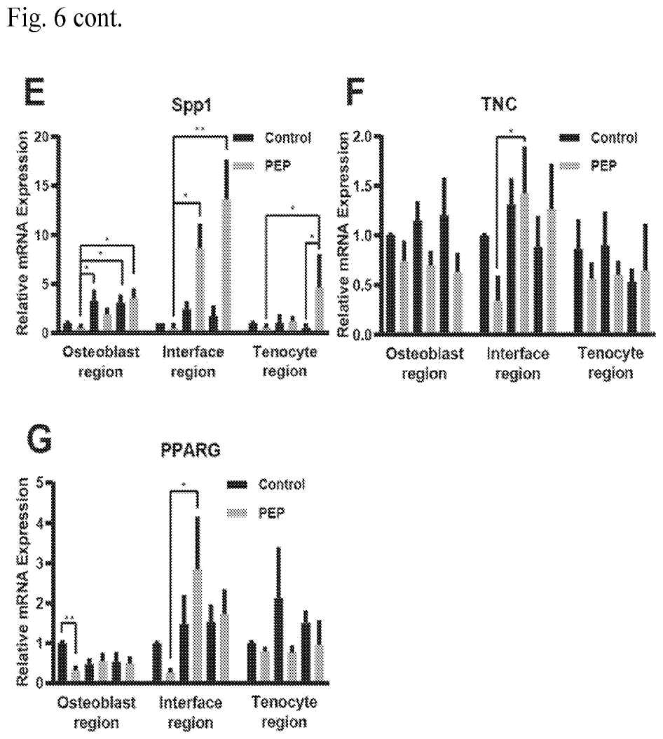

In the in vitro trial, mRNA levels of Col1, TNC, DCN, SCX, Spp1, and EGR significantly increased in the PEP group at day 9 compared to the PEP group at day 3 in the interface region (P<0.05). mRNA levels of Col3, TNC, Spp1, EGR, and PPARG significantly increased in the PEP group at day 6 compared to the PEP group at day 3 in the interface region (P<0.05). Also in the PEP group, co-culture with direct cell-cell contact increased the expression of Col3, TNC, Spp1, PPARG, and EGR compared to co-cultures without direct cell-cell contact (FIG. 6A-6G).

### In Vivo Rat Model

While described below in the context of an exemplary embodiment in which the damaged tendon-bone interface involves a rotator cuff tear, the methods described herein may be practiced to treat and/or repair the tendon-bone interface at any site in the body, regardless of whether the tendon-bone interface is a natural site (e.g., an insertion site or other natural enthesis) or is an artificial tendon-bone interface (e.g., a surgically constructed tendon-bone interface). Additionally, any suitable animal model may be used to measure the efficacy of the compositions and methods described herein on tendon-bone interface repair.

### Biomechanical Testing

Repair of the tendon-bone interface may be measured by changes to one or more mechanical properties of the tendon-bone connection, such as, but not limited to, maximum load, tensile load, and/or stiffness. In one or more embodiments, mechanical testing may be used to compare tendon-bone interface repair progression in animals treated with different compositions, e.g., to compare animals treated with and without PEP. In one or more embodiments, the compositions and methods described herein that include PEP may improve mechanical properties of a tendon-bone interface during healing compared to compositions and methods that do not include PEP.

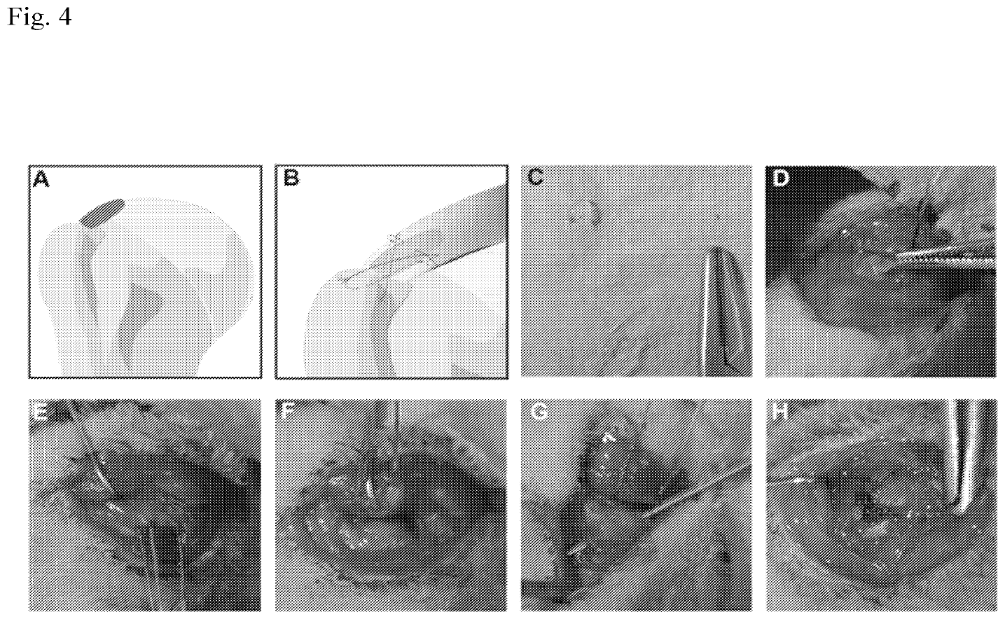

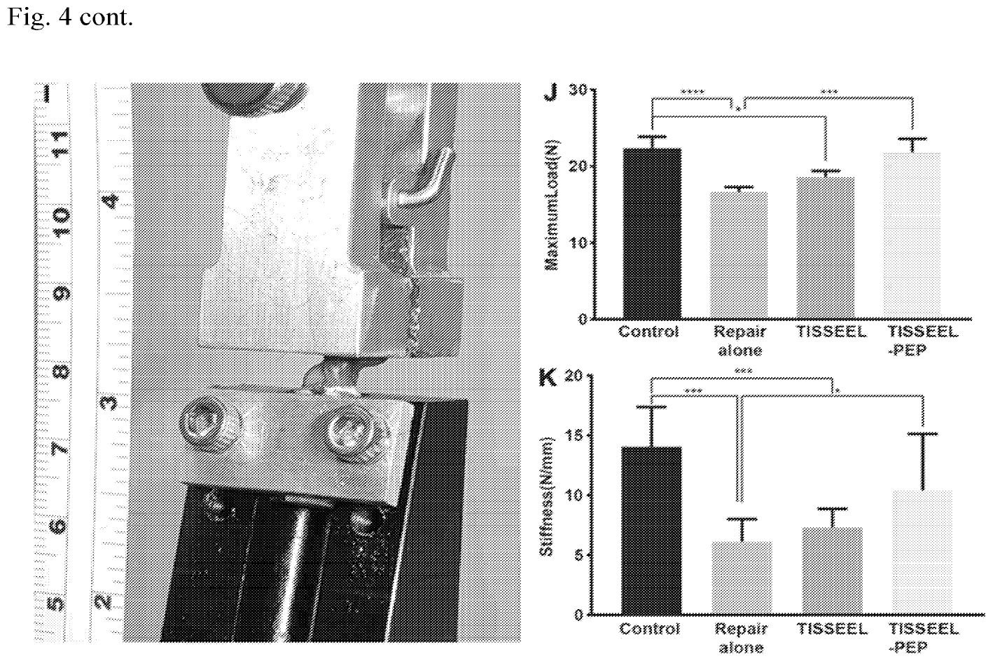

Maximum load of repaired rotator cuff at six weeks after surgery revealed no statistically significant difference between the TISSEEL-PEP and the normal, healthy control group. Maximum tensile load was 22.36 N+1.51 N (n=8) in the normal control group and 21.83 N 1.78 N (n=8) in the TISSEEL-PEP group. Compared with the other three groups, the maximum load of the repair-only group (16.63 N+0.67 N, n=8) was the lowest, which was statistically significant compared to both the TISSEEL-PEP group and the normal, healthy control group (P<0.01; FIG. 4J). The load in the TISSEEL group (18.62 N+0.77 N, n=8; P=0.03) fell between the repair-only group and the TISSEEL-PEP group and was significantly different from the normal, healthy control group (FIG. 4J). Evaluation of stiffness in the four groups is shown in FIG. 4K. The TISSEEL-PEP group exhibited a stiffness value of 10.41 N/mm ±4.71 N/mm, which showed similarities to the normal, healthy control group (14.06 N/mm+3.31 N/mm; P=0.11). Stiffness values were lowest in the repair-alone group, with the TISSEEL group producing stiffness values between the repair-alone group and the TISSEEL-PEP group.

### Histologic Analysis

Repair of a damaged tendon-bone interface may be measured by histological analysis of the damaged area. Histological properties that may be measured include, but are not limited to, inflammation, scar formation, collagen fiber arrangement, vascularity, and mineralization. In one or more embodiments, histologic analysis may be used to compare tendon-bone interface repair progression in animals treated with different compositions, e.g., to compare animals treated with and without PEP. In one or more embodiments, the compositions and methods described herein that include PEP may improve histologic measures of a tendon-bone interface during healing compared to compositions and methods that do not include PEP.

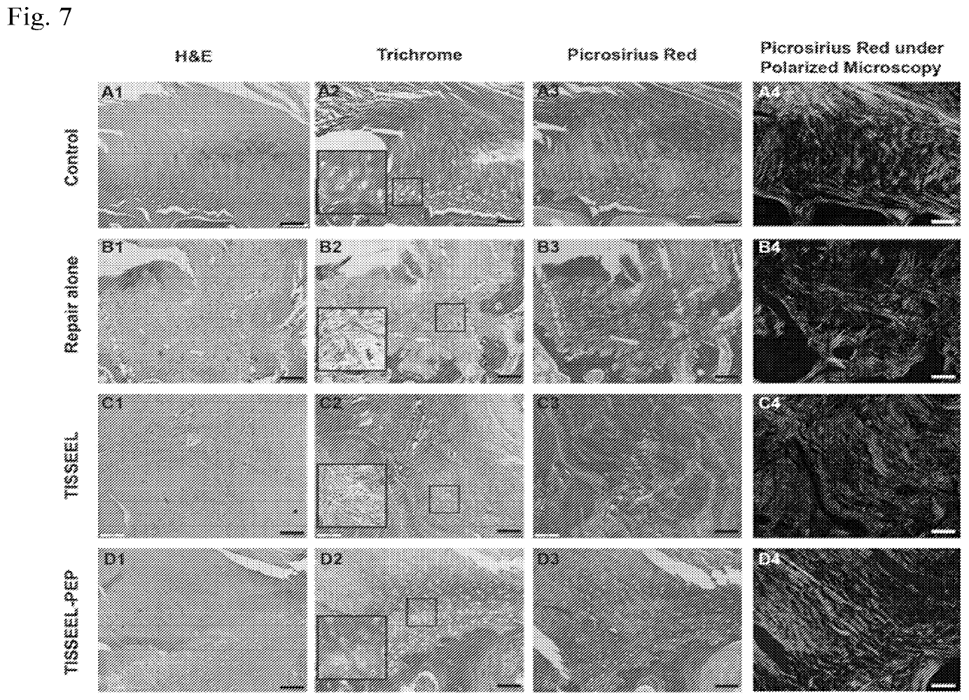

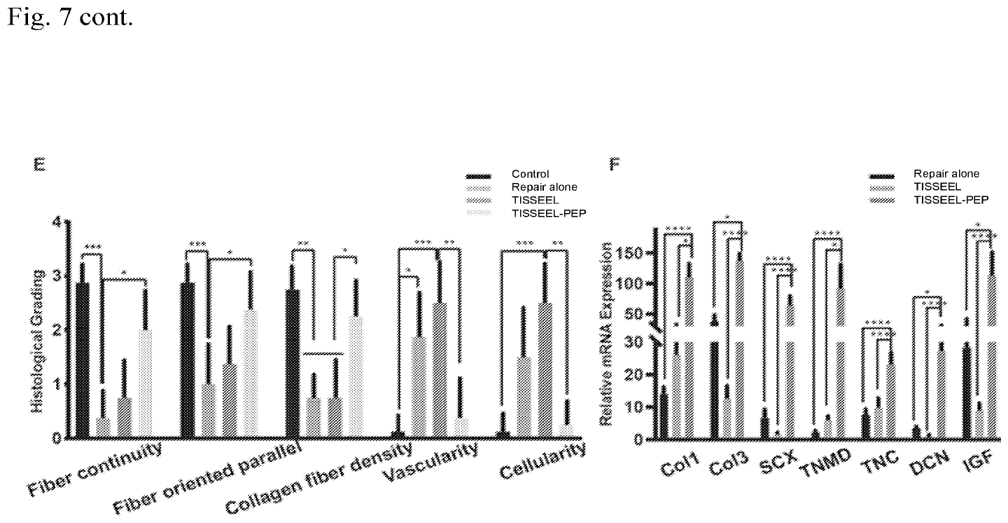

In the normal control group, highly aligned collagen fibers were interdigitated into mineralized fibrocartilage passing through the unmineralized fibrocartilage region. After six weeks, specimens in the TISSEEL-PEP group demonstrated that the tendon-bone interface had more organized and dense collagen fibers, fewer inflammatory cells, and vascularity, which was similar to the native interface. Furthermore, an appearance of natural enthesis was also observed in the TISSEEL-PEP group compared with the repair-alone and TISSEEL groups (all P<0.05) (FIG. 7).

In the repair-alone and TISSEEL groups, a mass of inflammatory cells, consisting primarily of polymorphonuclear leukocytes, was present. In addition, a looser, scar-like, and irregular meshwork of collagen fibers was observed in the repair-alone group. The TISSEEL group showed dense inflammatory cells and relatively organized alignment of collagen fibers and scar tissue with newly formed fibrovascular tissues at the tendon-bone interface compared to the repair-alone group (P<0.05) (FIG. 7).

Under polarized microscopy, collagen networks appeared to be shorter and thinner in the repair-alone and TISSEEL groups than in the normal control group and TISSEEL-PEP groups. In comparison with the normal control group, collagen fiber structure in the TISSEEL-PEP treated group showed restoration of fiber continuity, parallel orientation, and robust density that was comparable to that of the native control. The restoration of the native architecture in the TISSEEL-PEP group can be appreciated in the microscopy images (FIG. 7).

Based on these results, PEP with TISSEEL promotes remodeling of collagen fibers and new cartilage-like tissue at the tendon-bone interface after six weeks.

Changes of mRNA Levels

Repair of a damaged tendon-bone interface may be measured by changes in gene expression at the damaged area. Changes to gene expression may be measured by quantifying levels of messenger RNA (mRNA). Increased expression of multiple genes may indicate improved repair. Genes that may be quantified include Col1, Col3, SCX, Tnmd, TNC, DCN, and IGF. In one or more embodiments, gene expression levels may be used to compare tendon-bone interface repair progression in animals treated with different compositions, e.g., to compare animals treated with and without PEP. In one or more embodiments, the compositions and methods described herein that include PEP may increase expression of genes associated with repair of a tendon-bone interface during healing compared to compositions and methods that do not include PEP.

In the in vivo trial, the TISSEEL-PEP group showed a significant increase in expression of Col1, Col3, SCX, Tnmd, TNC, DCN, and IGF compared to all other groups (P<0.05) (FIG. 7F). Expressions of osteogenic-related genes (Spp1, Runx2) and chondrogenic genes (COMP and Co/2) were not detected.

The response to PEP in the tendon-bone interface was evaluated in this study. Treatment that included PEP in a rotator cuff model of repair at the tendon-bone interface resulted in significant improvement in biomechanical characteristics. Histologic observation revealed well-organized collagen fiber along with upregulated expression of tendon and tendon-bone-relative markers. The diversity of ingredients in exosomes leads to the multipotency of exosomes so that PEP exosomes accelerate the healing process in tendon-bone interface repair by acting on multiple targets and multiple pathways.

The strength of TISSEEL-PEP GROUP is close to normal, healthy rotator cuff strength. The histologic results show more organized and tighter collagenous tissue at the tendon-bone interface in the TISSEEL-PEP group. Thus, the TISSEEL-PEP treatment group exhibited strength and histological similarities with normal healthy rotator cuff compared to the other treatment groups (repair alone and TISSEEL alone). Further, the results of gene expression were confirmed through the biomechanical and histologic results. In addition to enhancing IGF expression, PEP was found to promote the upregulation of tendon-related genes (Col1, Col3, SCX, Tnmd, and DCN), leading to rearrangement of collagen and matrix constituents of the extracellular matrix during healing of the rotator cuff tendon-bone interface. PEP therefore is involved in releasing components that help remold the tendon-bone interface structure. Further, PEP and direct cell-cell contact were interlinked and reinforced each other in vitro, which may explain why the enthesis area of the rotator cuff had a higher recovery in vivo.

This disclosure therefore describes compositions and methods for improving repair of damaged tendon-bone interface. Generally, the compositions include PEP and a pharmaceutically acceptable carrier. In a surgical setting, the PEP may be combined with a carrier that is suitable for application to tendon tissue such as, for example, a surgical glue, a tissue adhesive, and/or a supportive matrix (e.g., a collagen scaffold, hydrogel, etc.).

Thus, the method includes administering an effective amount of the composition to tendon tissue in need of repair. In this aspect, an “effective amount” is an amount effective to increase osteoblast-tenocyte interface, improve at least one histological measure of the tendon-bone interface, increase expression of at least one gene that promotes repair of a damaged tendon-bone interface, or increase at least one biomechanical measure of the tendon-bone interface compared to osteoblast-tenocyte interface of a bone-tendon interface treated without PEP.

Exemplary histological measures include, but are not limited to, an increase in fiber continuity, an increase in fiber parallel orientation, an increase in collagen fiber density, a decrease in vascularity, or a decrease in cellularity compared to a bone-tendon interface treated without PEP.

In one or more embodiments, progression of tendon-bone repair can be measured by gene expression. For example, measuring expression of tendon-related genes, osteogenic-related genes, and/or chondrogenic genes can used to indicate the progression of repair to a damaged tendon-bone interface. Exemplary genes that promote repair of a damaged tendon-bone interface include, but are not limited to, type I fibrillar collagen (Col1), type III fibrillar collagen (Col3), scleraxis BHLH transcription factor (SCX), tenomodulin (TNMD), decorin (DCN), or insulin-like growth factor 1 (IGF-1) in tissues of the tendon-bone interface.

In one or more embodiments, repair to a damaged tendon-bone interface can be characterized using biomechanical measures. Exemplary biomechanical measures include, but are not limited to, maximum load or stiffness. Typically, as a damaged tendon-bone interface heals, the interface becomes stronger and maximum load and stiffness increase.

In one or more embodiments, the compositions and methods described herein may increase the rate of repair of tendon-bone damage.

As used herein, a “subject” can be a human or any non-human animal. Exemplary non-human animal subjects include, but are not limited to, a livestock animal or a companion animal. Exemplary non-human animal subjects include, but are not limited to, animals that are hominid (including, for example chimpanzees, gorillas, or orangutans), bovine (including, for instance, cattle), caprine (including, for instance, goats), ovine (including, for instance, sheep), porcine (including, for instance, swine), equine (including, for instance, horses), members of the family Cervidae (including, for instance, deer, elk, moose, caribou, reindeer, etc.), members of the family Bison (including, for instance, bison), feline (including, for example, domesticated cats, tigers, lions, etc.), canine (including, for example, domesticated dogs, wolves, etc.), avian (including, for example, turkeys, chickens, ducks, geese, etc.), a rodent (including, for example, mice, rats, etc.), a member of the family Leporidae (including, for example, rabbits or hares), members of the family Mustelidae (including, for example ferrets), or member of the order Chiroptera (including, for example, bats).

PEP may be formulated with a pharmaceutically acceptable carrier to form a pharmaceutical composition. As used herein, “carrier” includes any solvent, dispersion medium, vehicle, coating, diluent, antibacterial, and/or antifungal agent, isotonic agent, absorption delaying agent, buffer, hydrogel, carrier solution, suspension, colloid, water, and the like. The use of such media and/or agents for pharmaceutical active substances is well known in the art. Except insofar as any conventional media or agent is incompatible with PEP, its use in the therapeutic compositions is contemplated. Supplementary active ingredients also can be incorporated into the compositions. As used herein, “pharmaceutically acceptable” refers to a material that is not biologically or otherwise undesirable, i.e., the material may be administered to an individual along with the PEP without causing any undesirable biological effects or interacting in a deleterious manner with any of the other components of the pharmaceutical composition in which it is contained. As noted above, in a surgical setting, exemplary suitable carriers include surgical glue, tissue adhesive, or supportive matrix (e.g., a collagen scaffold). As used herein, “collagen scaffold” refers to a three-dimensional network that includes collagen, such as a hydrogel.

In one or more embodiments, the supportive matrix includes least one extracellular matrix component. Suitable extracellular matrix components include, but are not limited to, proteins such as collagen, elastin, fibronectin, or laminin, proteoglycans, and hyaluronic acid. In embodiments wherein the composition includes collagen, the collagen may be provided as procollagen, fibrillar collagen, such as type I collagen, type III collagen, or a combination thereof. In embodiments wherein the composition includes collagen, the collagen may be provided as a collagen scaffold. In one or more other embodiments, the extracellular matrix components may be supplied in any suitable form, such as purified recombinant protein.

A pharmaceutical composition containing PEP may be formulated in a variety of forms adapted to a preferred route of administration. Thus, a pharmaceutical composition can be administered via known routes including, for example, oral, parenteral (e.g., intradermal, transcutaneous, subcutaneous, intramuscular, intravenous, intraperitoneal, etc.), or topical (e.g., application to tendon tissue exposed during surgery, intranasal, intrapulmonary, intramammary, intravaginal, intrauterine, intradermal, transcutaneous, rectally, etc.). A pharmaceutical composition can be administered to a mucosal surface, such as by administration to, for example, the nasal or respiratory mucosa (e.g., by spray or aerosol). A pharmaceutical composition also can be administered via a sustained or delayed release.

Thus, a pharmaceutical composition may be provided in any suitable form including but not limited to a solution, a suspension, an emulsion, a spray, an aerosol, or any form of mixture. The pharmaceutical composition may be delivered in formulation with any pharmaceutically acceptable excipient, carrier, or vehicle. For example, the formulation may be delivered in a conventional topical dosage form such as, for example, a cream, an ointment, an aerosol formulation, a non-aerosol spray, a gel, a lotion, and the like. In embodiments wherein the formulation is a gel, the gel may have any suitable density. For example, for certain embodiments in a surgical setting, the pharmaceutical composition may be formulated as a gel having sufficient density to keep the formulation in a desired location. The formulation may further include one or more additives including such as, for example, an adjuvant, a skin penetration enhancer, a colorant, a fragrance, a flavoring, a moisturizer, a thickener, and the like.

Suitable excipients may include, for example, human or bovine collagen, hyaluronic acid-based compounds, human fibrinogen, or human thrombin.

In one or more embodiments, the compositions described herein may be lypophilized. The lyophilized composition including PEP may be combined with an additional excipient, which may additionally be lyophilized. Components of the lyophilized composition may be co-packaged or may be separately provided and mixed before use to create a PEP-loaded biocompatible scaffold. The lyophilized excipient may be, for example, lyophilized human or bovine collagen, hyaluronic acid-based compounds, human fibrinogen, human thrombin, or other lyophilized powders that form a biocompatible gel when put in contact with bodily fluids (ex. blood or interstitial fluid).

In one or more embodiments, a composition described herein are administered via injection into/onto the tendon-bone interface, arthroscopically, or during open surgical repair. A composition as described herein may be administered alone or in addition to traditional surgical repair methods, such as sutures or staples. A composition as described herein also may also be used to enhance the biocompatibility and therapeutic effect of tendon sutures, anchors, patches, or other devices used to repair tendinous injures.

A formulation may be conveniently presented in unit dosage form and may be prepared by methods well known in the art of pharmacy. Methods of preparing a composition with a pharmaceutically acceptable carrier include the step of bringing the PEP into association with a carrier that constitutes one or more accessory ingredients. In general, a formulation may be prepared by uniformly and/or intimately bringing the PEP into association with a liquid carrier, a finely divided solid carrier, or both, and then, if necessary, shaping the product into the desired formulations.

The amount of PEP administered can vary depending on various factors including, but not limited to, the content and/or source of the PEP being administered, the weight, physical condition, and/or age of the subject, and/or the route of administration. Thus, the absolute weight of PEP included in a given unit dosage form can vary widely, and depends upon factors such as the species, age, weight, and physical condition of the subject, and/or the method of administration. Accordingly, it is not practical to set forth generally the amount that constitutes an amount of PEP effective for all possible applications. Those of ordinary skill in the art, however, can readily determine the appropriate amount with due consideration of such factors.

In one or more embodiments, a dose of PEP can be measured in terms of the PEP exosomes delivered in a dose. Thus, in one or more embodiments, the method can include administering sufficient PEP to provide a dose of, for example, from 1×10⁶ PEP exosomes to 1×10¹⁵ PEP exosomes to the subject, although in one or more embodiments the methods may be performed by administering PEP in a dose outside this range.

In one or more embodiments, therefore, the method can include administering sufficient PEP to provide a minimum dose of at least 1×10⁶ PEP exosomes, at least 1×107 PEP exosomes, at least 1×10⁸ PEP exosomes, at least 1×109 PEP exosomes, at least 1×10¹⁰ PEP exosomes, at least 1×10¹¹ PEP exosomes, at least 2×10¹¹ PEP exosomes, at least 3×10¹¹ PEP exosomes, at least 4×10¹¹ PEP exosomes, at least 5×10¹¹ PEP exosomes, at least 6×10¹¹ PEP exosomes, at least 7×10¹¹ PEP exosomes, at least 8×10¹¹ PEP exosomes, at least 9×10¹¹ PEP exosomes, at least 1×10¹² PEP exosomes, 2×10¹² PEP exosomes, at least 3×10¹² PEP exosomes, at least 4×10¹² PEP exosomes, or at least 5×10¹² PEP exosomes, at least 1×10¹³ PEP exosomes, or at least 1×10¹⁴ PEP exosomes.

In one or more embodiments, the method can include administering sufficient PEP to provide a maximum dose of no more than 1×10¹⁵ PEP exosomes, no more than 1×10¹⁴ PEP exosomes, no more than 1×10¹³ PEP exosomes, no more than 1×10¹² PEP exosomes, no more than 1×10¹¹ PEP exosomes, or no more than 1×10¹⁰ PEP exosomes.

In one or more embodiments, the method can include administering sufficient PEP to provide a dose characterized by a range having endpoints defined by any a minimum dose identified above and any maximum dose that is greater than the minimum dose. For example, in one or more embodiments, the method can include administering sufficient PEP to provide a dose of from 1×10¹¹ to 1×10¹³ PEP exosomes such as, for example, a dose of from 1×10¹¹ to 5×10¹² PEP exosomes, a dose of from 1×10¹² to 1×10¹³ PEP exosomes, or a dose of from 5×10¹² to 1×10¹³ PEP exosomes. In certain embodiments, the method can include administering sufficient PEP to provide a dose that is equal to any minimum dose or any maximum dose listed above. Thus, for example, the method can involve administering a dose of 1×10¹⁰ PEP exosomes, 1×10¹¹ PEP exosomes, 5×10¹¹ PEP exosomes, 1×10¹² PEP exosomes, 5×10¹² PEP exosomes, 1×10¹³ PEP exosomes, or 1×10¹⁴ PEP exosomes.

Alternatively, a dose of PEP can be measured in terms of the concentration of PEP upon reconstitution from a lyophilized state. Thus, in one or more embodiments, the methods can include administering PEP to a subject at a dose of, for example, from a 0.01% solution to a 100% solution to the subject, although in one or more embodiments the methods may be performed by administering PEP in a dose outside this range. As used herein, a 100% solution of PEP refers to one vial of PEP (2×10¹¹ exosomes or 75 mg) solubilized in 1 ml of a liquid or gel carrier (e.g., water, phosphate buffered saline, serum free culture media, surgical glue, tissue adhesive, etc.). For comparison, a dose of 0.01% PEP is roughly equivalent to a standard dose of exosomes prepared using conventional methods of obtaining exosomes such as exosome isolation from cells in vitro using standard cell conditioned media.

In one or more embodiments, therefore, the method can include administering sufficient PEP to provide a minimum dose of at least 0.01%, at least 0.05%, at least 0.1%, at least 0.25%, at least 0.5%, at least 1.0%, at least 2.0%, at least 3.0%, at least 4.0%, at least 5.0%, at least 6.0%, at least 7.0%, at least 8.0%, at least 9.0%, at least 10%, at least 15%, at least 20%, at least 25%, at least 30%, at least 35%, at least 40%, at least 45%, at least 50%, at least 60%, or at least 70%.

In one or more embodiments, the method can include administering sufficient PEP to provide a maximum dose of no more than 100%, no more than 90%, no more than 80%, no more than 70%, no more than 60%, no more than 50%, no more than 40%, no more than 30%, no more than 20%, no more than 10%, no more than 9.0%, no more than 8.0%, no more than 7.0%, no more than 6.0%, no more than 5.0%, no more than 4.0%, no more than 3.0%, no more than 2.0%, no more than 1.0%, no more than 0.9%, no more than 0.8%, no more than 0.7%, no more than 0.6%, no more than 0.5%, no more than 0.4%, no more than 0.3%, no more than 0.2%, or no more than 0.1%.

In one or more embodiments, the method can include administering sufficient PEP to provide a dose characterized by a range having endpoints defined by any a minimum dose identified above and any maximum dose that is greater than the minimum dose. For example, in one or more embodiments, the method can include administering sufficient PEP to provide a dose of from 1% to 50% such as, for example, a dose of from 5% to 20%. In certain embodiments, the method can include administering sufficient PEP to provide a dose that is equal to any minimum dose or any maximum dose listed above. Thus, for example, the method can involve administering a dose of 0.05%, 0.25%, 1.0%, 2.0%, 5.0%, 20%, 25%, 50%, 80%, or 100%.

A single dose may be administered all at once, continuously for a prescribed period of time, or in multiple discrete administrations. When multiple administrations are used, the amount of each administration may be the same or different. For example, a prescribed daily dose of may be administered as a single dose, continuously over 24 hours, as two administrations, which may be equal or unequal. When multiple administrations are used to deliver a single dose, the interval between administrations may be the same or different. In one or more certain embodiments, PEP may be administered from a one-time administration, for example, during a surgical procedure.

In one or more certain embodiments in which multiple administrations of the PEP composition are administered to the subject, the PEP composition may be administered as needed to heal and/or repair the tendon-bone interface to the desired degree. Alternatively, the PEP composition may be administered twice, three times, four times, five times, six times, seven times, eight times, nine times, or at least ten times. The interval between administrations can be a minimum of at least one day such as, for example, at least three days, at least five days, at least seven days, at least ten days, at least 14 days, or at least 21 days. The interval between administrations can be a maximum of no more than six months such as, for example, no more than three months, no more than two months, no more than one month, no more than 21 days, or no more than 14 days.

In one or more embodiments, the method can include multiple administrations of PEP to at an interval (for two administrations) or intervals (for more than two administrations) characterized by a range having endpoints defined by any a minimum interval identified above and any maximum interval that is greater than the minimum interval. For example, in one or more embodiments, the method can include multiple administrations of PEP at an interval or intervals of from one day to six months such as, for example, from three days to ten days. In one or more certain embodiments, the method can include multiple administrations of PEP at an interval of that is equal to any minimum interval or any maximum interval listed above. Thus, for example, the method can involve multiple administrations of PEP at an interval of three days, five days, seven days, ten days, 14 days, 21 days, one month, two months, three months, or six months.

In one or more embodiments, the methods can include administering a cocktail of PEP that is prepared from a variety of cell types, each cell type having a unique tendon-bone interface repairing profile—e.g., protein composition and/or gene expression. In this way, the PEP composition can provide a broader spectrum of tendon-bone interface repair than if the PEP composition is prepared from a single cell type.

In the preceding description and following claims, the term “and/or” means one or all of the listed elements or a combination of any two or more of the listed elements; the terms “comprises,” “comprising,” and variations thereof are to be construed as open ended i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Reference throughout this specification to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments. Furthermore, the particular features, configurations, compositions, or characteristics may be combined in any suitable manner in one or more embodiments. Thus, features described in the context of one embodiment may be combined with features described in the context of a different embodiment except where the features are necessarily mutually exclusive.

For any method disclosed herein that includes discrete steps, the steps may be conducted in any feasible order. And, as appropriate, any combination of two or more steps may be conducted simultaneously.

As used herein, the terms “preferred” and “preferably” refer to embodiments of the invention that may afford certain benefits under certain circumstances. However, other embodiments may also be preferred under the same or other circumstances. Furthermore, the recitation of one or more preferred embodiments does not imply that other embodiments are not useful and is not intended to exclude other embodiments from the scope of the invention.

The present invention is illustrated by the following examples. It is to be understood that the particular examples, materials, amounts, and procedures are to be interpreted broadly in accordance with the scope and spirit of the invention as set forth herein.

### EXEMPLARY EMBODIMENTS

Embodiment 1 is a method of repairing a damaged bone-tendon interface in a subject, the method comprising:
contacting the damaged bone-tendon interface with an effective amount of a composition comprising:
   purified exosome product (PEP); and
   a pharmaceutically acceptable carrier.

Embodiment 2 is the method of embodiment 1, wherein the PEP comprises spherical or spheroid exosomes having a diameter no greater than 300 nm.

Embodiment 3 is the method of embodiment 1, wherein the PEP comprises spherical or spheroid exosomes having a diameter of from 56 nm to 151 nm.

Embodiment 4 is the method of any preceding embodiment, wherein the PEP comprises spherical or spheroid exosomes having a mean diameter of 97 nm.

Embodiment 5 is the method of embodiment 4, wherein the PEP comprises spherical or spheroid exosomes having a mean diameter of 97 nm+54 nm.

Embodiment 6 is the method of any preceding embodiment, wherein the PEP comprises: from 1% to 20% CD63⁻ exosomes; and from 80% to 99% CD63⁺ exosomes.

Embodiment 7 is the method of any one of embodiments 1-5, wherein the PEP comprises at least 50% CD63⁻ exosomes.

Embodiment 8 is the method of any preceding embodiment, wherein the PEP comprises from 1×10¹¹ PEP exosomes to 1×10¹³ PEP exosomes.

Embodiment 9 is the method of embodiment 8, wherein the PEP comprises from 1×10¹² PEP exosomes to 1×10¹³ PEP exosomes.

Embodiment 10 is the method of any preceding embodiment, wherein the composition further comprises a supportive matrix.

Embodiment 11 is the method of embodiment 10, wherein the supportive matrix comprises a collagen scaffold.

Embodiment 12 is the method of embodiment 10, wherein the supportive matrix comprises a tissue sealant or a fibrin sealant.

Embodiment 13 is the method of any preceding embodiment, wherein an effective amount is an amount effective to increase osteoblast-tenocyte interface compared to osteoblast-tenocyte interface of a bone-tendon interface treated without PEP.

Embodiment 14 is the method of any preceding embodiment, wherein an effective amount is an amount effective to improve at least one histological measure of the tendon-bone interface compared to a bone-tendon interface treated without PEP.

Embodiment 15 is the method of embodiment 14, wherein the histological measure comprises an increase fiber continuity, an increase fiber parallel orientation, an increase collagen fiber density, a decrease vascularity, or a decrease cellularity compared to a bone-tendon interface treated without PEP.

Embodiment 16 is the method of any preceding embodiment, wherein an effective amount is an amount effective to increase expression of at least one gene that promotes repair of a damaged tendon-bone interface.

Embodiment 17 is the method of embodiment 16, wherein the gene encodes type I fibrillar collagen (Col1), type III fibrillar collagen (Col3), scleraxis BHLH transcription factor (SCX), tenomodulin (TNMD), decorin (DCN), or insulin-like growth factor 1 (IGF-1) in tissues of the tendon-bone interface.

Embodiment 18 is the method of any preceding embodiment, wherein an effective amount is an amount effective to increase at least one biomechanical measure of the tendon-bone interface compared to a bone-tendon interface treated without PEP.

Embodiment 19 is the method of embodiment 18, wherein the biomechanical measure comprises maximum load or stiffness.

Embodiment 20 is the method of any preceding embodiment, wherein:
the damaged bone-tendon interface comprises complete separation of tendon from bone; and
the method further comprises surgically reattaching the tendon to the bone.

Embodiment 21 is the method of any preceding embodiment wherein:
the damaged tendon-bone interface comprises partial separation of tendon from bone; and
the method comprises implanting the PEP composition at a site effective for contacting the PEP composition with the damaged tendon-bone interface.

### EXAMPLES

### Example 1

In this Example, transmission electron microscopy was used to measure and count PEP particles.

### Transmission Electron Microscopy

Transmission electron microscopy (TEM) observation was performed with a transmission electron microscope (JEM-1400Plus 120 kV Transmission Electron Microscope, JEOL Ltd., Tokyo, Japan). Before the TEM test, a vial of sealed PEP was mixed with 1 mL PBS (Gibco, Thermo Fisher Scientific, Inc., Waltham, MA) to prepare the 100% (vol/vol) PEP solution. 50 L of the PEP solution was transferred to a microcentrifuge tube and 1 mL 2.5% glutaraldehyde (pH 7.0) in 0.1 M sodium cacodylate solution was added, then mixed for one hour at 4° C. Fixed samples were washed in sodium cacodylate buffer (pH 7.4) three times for 10 minutes each. Next, samples were post-fixed in 2% osmium tetroxide for one hour at 4° C. and washed in buffer, dehydrated, and stained with 2% uranyl acetate according to the standard protocol. PEP (50-μL sample per grid) was examined under a transmission electron microscope at 80 kV and electron micrographs were taken.

### Nanoparticle Tracking Analysis

Size distribution and concentration of PEP were determined using a nanoparticle tracking characterization system (NS300, NanoSight Ltd., Malvern, United Kingdom). The PEP solutions (100%, vol/vol) were diluted 1,000 times with 1 mL PBS diluent before loading into the sample chambers. PEP concentration, mean, and mode PEP size were analyzed using NTA 3.2 analytical software (NanoSight Ltd., Malvern, United Kingdom).

### Example 2

In this Example, a cell culture model was used to study the impact of PEP on tendon-bone interface repair.

### Preparation of PEP and Fibrin Sealant (TISSEEL) with and without PEP

PEP was obtained from the API at the Mayo Clinic Center for Regenerative Medicine. The product was formulated and stored in a stabilized lyophilized powder form in vials to allow for room temperature storage until processing (FIG. 2A).

Fibrin sealant is a biodegradable pulp-like tissue that can be used as a drug delivery vehicle, and is very effective at achieving a local and sustained release of exosomes. In this Example, TISSEEL kit, a fibrin sealant product, was used either with or without added PEP. Before preparation of the TISSEEL kit (Baxter International, Inc., Deerfield, IL), a vial of sealed PEP powder was mixed with 1 mL PBS (Gibco, Thermo Fisher Scientific, Inc., Waltham, MA) to prepare the 100% (vol/vol) PEP solution. 400 μL PEP solution was added into the 600 μL CaCl₂) solution (one of the contents of the TISSEEL kit), and the solution was normalized to a 40% (vol/vol) concentration. Manufacturer directions were then followed to finish kit preparation (FIG. 2B). The final concentration of PEP in TISSEEL was 20% (vol/vol). The gel was manually cut into small cubes (3×3×3 mm). In the co-culture model, a single cube was placed into the small hole in one well of a six-well plate. The culture medium with PEP gel was used to simulate the PEP microenvironment in vivo. For osteogenic induction, the medium with PEP as described above was used as the positive control group. For the in vivo trial, the cube was placed directly on the RC repair site, between the supraspinatus tendon and the greater tuberosity.

### Primary Cells, Culture Conditions, and Identification

Primary osteoblasts were isolated from the calvaria of neonatal rats that had been euthanized—a process which did not affect the osteoblasts—under InstitutionalAnimal Care and Use Committee (IACUC)—approved guidelines using methods described previously (Liu et al., 2019, Cell and Tissue Banking 20:173-182). To harvest osteoblasts, calvaria segments were first immersed in a mixture of 0.1% (wt/vol) collagenase I and 0.05% trypsin with 0.004% ethylenediaminetetraacetic acid for 60 minutes. Cells were then harvested from the third to fifth immersions and cultured in minimum essential medium a (Invitrogen, Thermo Fisher Scientific, Inc., Waltham, MA) supplemented with 10% fetal bovine serum (Gibco, Thermo Fisher Scientific, Inc., Waltham, MA) and 1% penicillin-streptomycin (Gibco, Thermo Fisher Scientific, Inc., Waltham, MA) at 37° C. and 5% C02. To test the osteogenic potential of the primary osteoblasts and PEP, we established three groups: The above medium served as the negative control group; the above medium supplemented with PEP served as the positive control group; and the StemPro Osteogenesis Differetiation Kit (Thermo Fisher Scientific, Inc., Waltham, MA) served as the osteogenic inductive group.

Osteoblasts were identified with alkaline phosphatase (ALP) staining. After being cultured for seven or 14 days, the primary osteoblasts were washed twice with cold phosphate-buffered saline (PBS), fixed with 4% paraformaldehyde for 30 minutes, rinsed with deionized water, and stained with an ALP staining kit (Abcam, Cambridge, United Kingdom) for 30 minutes under protection from direct light, according to the manufacturer's instructions. Images were then obtained with a Nikon camera (Nikon, Minato City, Japan).

Primary tenocytes were isolated from eight-week-old female Sprague-Dawley rats euthanized under IACUC-approved guidelines, using methods described previously (Zhang et al., 2010, BMC MusculoskeletalDisorders 11:10). Rat flexor tendons were harvested and the paratenon sheath layer was separated by gentle scraping. Tendons were washed three times with sterile PBS, cut into small segments, and cultivated with conditioned medium as described above until confluent growth occurred. The cell culture medium was refreshed every three days. Cells from the third to sixth passages were used for all trials. The tenocytes were identified by detection of tendon-specific genes: collagen type 1 (Col1), collagen type 3 (Col3), and Scleraxis (SCX) expression. For quantitative reverse transcriptase-polymerase chain reaction (RT-PCR), the value of each mRNA expression was normalized by glyceraldehyde 3-phosphate dehydrogenase (GAPDH) mRNA expression; the rat primary osteoblasts served as the control group.

### Co-Culture Model

An in vitro co-culture model was used based on a previously described model (Bogdanowicz et al., 2014, Methods in Molecular Biology 1202:29-36). The co-culture model used a 6-well plate and cryopreservation tube. In addition to the osseous, interface, and tenocyte regions, the new model had an additional point of entry for a drug vehicle. Cell culture grade agarose (Sigma-Aldrich, St. Louis, MO) was poured into the 6-well plate and cut into three holes using the cryopreservation tube, leaving a divider of 3 mm in width. The model was then transferred to a new 6-well plate and fixed with uncoagulated agarose to the bottom of the plate (FIG. 1A-lE). The rat primary osteoblasts were seeded on the slightly larger hole to the left, and tenocytes on the slightly larger hole to the right. The small center hole held a 20% PEP with TISSEEL cube (FIG. 1A). After allowing 30 minutes for cell attachment, culture medium was added until the model was nearly submerged. Co-cultures were incubated for two days, the divider between the two cell populations was cut, and the PEP vehicle was added into the small hole. No drugs were added to the control group. Culture medium overflowed the model and was changed every three days. Cell migration into the interface region was recorded twice daily using the IncuCyte HD system (IncuCyte ZOOM, Essen BioScience Inc., Ann Arbor, MI). The cell boundary was manually traced using Photoshop CS6 (Adobe, San Jose, CA). For the PCR test, the model was moved and washed with ice-cold PBS at three days after direct contact of the two cell populations. Cells were then detached from the plate with cell scrapers in their respective regions and stored with TRIzol (TRI Reagent, Sigma-Aldrich, St. Louis, MO) in tubes, which were stored at −80° C. for the PCR test. Results are shown in FIG. 5 and described in greater detail herein.

### Example 3

In this Example, a rat model of rotator cuff injury was used to compare rates of repair when animals were treated with sutures only, sutures and TISSEL, or sutures, TISSEL, and PEP.

### Animal Study Design and Rat RCT Model

Thirty-six Sprague-Dawley rats (adult females, 4-5 months old, 258-552 g weight) were used. They were randomly divided into three groups, and right shoulders were used as the surgical sides (n=12 for all groups): repair-alone group; repair+TISSEEL (Baxter International, Inc., Deerfield, IL) (TISSEEL group); and repair+TISSEEL+PEP (TISSEEL-PEP group). Rats were anesthetized with 2%-3% isoflurane in 2 L/min 100% oxygen delivered via mask until disappearance of the toe-pinch reflex via induction chamber. An intramuscular injection of meloxicam (1 mg/kg) was given as preemptive analgesia. Each rat was placed on a warm plate to maintain body temperature and reduce the risk of hypothermia. Anesthesia was maintained with a continuous flow of 1.5%-2% isoflurane in 1 L 100% oxygen mixture via nose cone.

The surgical site was scrubbed with 2% chlorhexidine gluconate, and the skin was incised with a sterile #15 scalpel blade in a transverse direction, 1 cm outside the deltoid muscle. The supraspinatus tendon from the subscapularis tendon anteriorly and the infraspinatus tendon posteriorly were identified and separated. The supraspinatus tendon was then transected at its insertion site on the greater tuberosity. To fresh the insertion site, tendon fibers were scraped at the insertion site with a scalpel. Then, one end of the double-armed 5-0 suture (ETHIBOND, Ethicon Inc., Raritan NJ) was passed through the tendon transversely, and small loops were made on both sides of the tendon using the modified Mason-Allen suture technique (FIGs. 4B, 4E, and 4F). A 0.5-mm hole was drilled transversely in the anterior-posterior direction through the proximal part of the humerus, and the other end of the suture was passed through the 0.5-mm hole (FIG. 4G). TISSEEL (Baxter International, Inc., Deerfield, IL, with or without seeded PEP, was placed on the repair site before tying the suture to the tendon at its insertion point on the greater tuberosity (FIGS. 4A, 4C, and 4D). TISSEEL with or without PEP was prepared as described in EXAMPLE 2. The detached deltoid muscle was repaired with a 4-0 polyglactin 910 suture (VICRYL, Ethicon, Inc., Raritan, NJ), and the skin with a 3-0 polyglactin 910 suture (VICRYL, Ethicon, Inc., Raritan, NJ) (FIG. 4H). A water-ibuprofen mixture (15 mg/kg) was administered daily for one week postoperatively in all groups. These doses were recommended by a laboratory animal veterinarian and approved in the IACUC protocol.

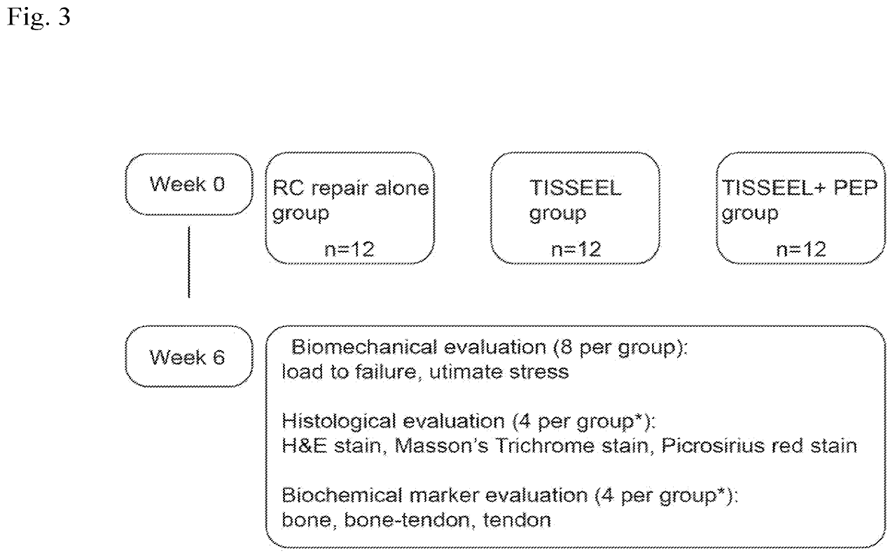

Six weeks after surgery, rats were euthanized via CO₂ asphyxiation. Eight rats from each group were used for biomechanical testing, and four rats from each group were used for both histologic analysis and qPCR measurement of mRNA expression. The left shoulders served as the normal control group (FIG. 3).

### Biomechanical Testing of RCT Repair

At six weeks post treatment, rats were euthanized by carbon dioxide inhalation to evaluate tissue healing. The peritendinous tissue of the supraspinatus tendon and the humerus was then removed completely with surgical loupes. After embedding the humerus in polymethylmethacrylate in a custom-designed fixture and holding the proximal end of the tendon in a spring-loaded clamp custom built for testing, the specimens were subjected to a preload of 0.2 N and preconditioned for five cycles of 0.1 mm displacement at a rate of 0.1 mm/s, then tested until failure under uniaxial tension at a rate of 0.1 mm/s (FIG. 4I). Finally, ultimate load to failure and stiffness were calculated from the force-displacement curve generated by a custom MATLAB program (MathWorks, Natick, MA).

### Histologic Analysis

After euthanasia at six weeks, the repair site at the supraspinatus tendon and its bony insertion was carefully dissected in each group. Specimens were fixed overnight with 10% formaldehyde and then placed in 14% ethylenediaminetetraacetic acid. Specimens were placed in 30%, 50%, and 70% ethanol alcohol for at least 30 minutes each before submission to the core laboratory for paraffin embedding. All specimens were embedded in a tissue embedding matrix (TISSUE-TEK, Sakura-Finetek, Ltd., Tokyo Japan) and cut into coronal sections (10 μm thick) with a cryostat (Leica Biosystems GmbH, Wetzlar, Germany). Histologic changes were analyzed with hematoxylin-eosin staining, Masson trichrome staining, and Picrosirius red staining. Picrosirius red stained tissue slices were observed by polarized light microscopy (BH2, Olympus Corp., Shinjuku, Japan). Representative microscopic images are shown in FIG. 7.

Collagen fiber continuity, parallel orientation, density, vascularity, and cellularity at the tendon-bone interface were assessed. Histologic findings were evaluated using a semiquantitative scoring system (0-3 grades per item). For collagen fiber continuity and collagen fibers oriented parallel to each other, scoring was defined by percentage: 0=0%-25% of proportion; 1=25%-50% of proportion; 2=50%-75% of proportion; and 3=75%-100% of proportion. For collagen fiber density, scoring was defined by percentage: 0=very loose, 1=loose, 2=dense, and 3=very dense. For vascularity and cellularity, scoring was defined by percentage: 0=absent or minimally present, 1=mildly present, 2=moderately present, and 3=severe or markedly present. Each slide was examined under a microscope (Olympus Corp., Shinjuku, Japan) and analyzed using ImageJ software (Schneider et al., 2012, Nature Methods 9(7), 671-675). Four samples were assessed by two independent observers for each group. Histological quantification is shown in FIG. 7E.

### RNA Isolation and Quantitative PCR

In the in vitro trial, after reaching the measurement time points, cells in corresponding regions (osteoblast region, tenocyte region, and interface region) were washed with PBS and detached by scraping separately. In the in vivo trial, tendon-bone tissues were dissected and flash-frozen in liquid nitrogen, then crushed with an abrasive tool. After homogenization, total RNA was extracted and purified using an RNA isolation kit (TRIZOL Plus, Invitrogen, Thermo Fisher Scientific, Inc., Waltham, MA). Total RNA was then quantitated using a spectrophotometer (NANODROP 1000, Thermo Fisher Scientific, Inc., Waltham, MA) and cDNA synthesis (RT-PCR) was performed using a cDNA synthesis kit (ISCRIPT, Bio-Rad Laboratories, Inc., Hercules, CA). Total RNA (1 μg) was reverse transcribed to complementary DNA using a kit (THERMOSCRIPT, Invitrogen, Thermo Fisher Scientific, Inc., Waltham, MA). Real-time PCR was performed in triplicate. Briefly, total RNA was extracted from cells using TRIzol reagent (Invitrogen, Thermo Fisher Scientific, Inc., Waltham, MA) according to the manufacturer's instructions. Complementary DNA (cDNA) was synthesized from equal amounts of RNA (1 μg) using the iScript cDNA Synthesis Kit (Bio-Rad Laboratories, Inc., Hercules, CA). All reactions were performed using SYBR Green PCR Master Mix (Quantabio, Beverly, MA) on a C1000 Touch Thermal Cycler (Bio-Rad Laboratories, Inc., Hercules, CA). Data from target genes were normalized to GAPDH and then calculated using the 2^{-ΔCt} formula with reference to the basal controls. For detection of gene marker expression, primers for tenocyte-related gene markers (Col1, Col3, SCX, tenomodulin (Tnmd), EGR1 (early growth response protein 1), decorin (DCN)), osteoblast-related gene markers (secreted phosphoprotein 1 (Spp1), tenascin C (TNC), RUNX family transcription factor 2 (Runx2), insulin-like growth factor 1 (IGF-I)), lipid metabolic related gene marker (peroxisome proliferator-activated receptor gamma (PPARG)) and chondrogenic related gene markers (Col2, cartilage oligomeric matrix protein (COMP)) were performed. Primers used in qPCR experiments were as previously described (Shi et al., 2021, J Orthop Res. 39(8):1825-1837. doi:10.1002/jor.24859).

### Statistical Analysis

Data were presented as mean (SD). Each trial was performed independently at least 3 times. Kruskal-Wallis one-way analysis of variance test with Dunn test were used for determining statistical significance for 2-group comparisons and multiple-group comparisons, respectively. Statistical comparisons between two groups were analyzed by nonpaired Student t test or Mann-Whitney test. All statistical tests were performed using GraphPad Prism 8 (GraphPad Software Inc., San Diego, CA). These data are shown in FIG. 7F. Results with asterisks were considered statistically significant (* P<0.05, ** P<0.01, *** P<0.001, **** P<0.0001).

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “approximately” or “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. A method of repairing a damaged bone-tendon interface in a subject, the method comprising:
contacting the damaged bone-tendon interface with an effective amount of a composition comprising:
purified exosome product (PEP); and
a pharmaceutically acceptable carrier.

2. The method of claim 1, wherein the PEP comprises spherical or spheroid exosomes having a diameter no greater than 300 nm.

3. The method of claim 1, wherein the PEP comprises spherical or spheroid exosomes having a diameter of from 56 nm to 151 nm.

4. The method of claim 1, wherein the PEP comprises spherical or spheroid exosomes having a mean diameter of 97 nm.

5. The method of claim 4, wherein the PEP comprises spherical or spheroid exosomes having a mean diameter of 97 nm±54 nm.

6. The method of claim 1, wherein the PEP comprises:
from 1% to 20% CD63⁻ exosomes; and
from 80% to 99% CD63'⁰ exosomes.

7. The method of claim 1, wherein the PEP comprises at least 50% CD63⁻ exosomes.

8. The method of claim 1, wherein the PEP comprises from 1×10¹¹ PEP exosomes to 1×10¹³ PEP exosomes.

9. The method of claim 8, wherein the PEP comprises from 1×10¹² PEP exosomes to 1×10¹³ PEP exosomes.

10. The method of claim 1, wherein the composition further comprises a supportive matrix.

11. The method of claim 10, wherein the supportive matrix comprises a collagen scaffold.

12. The method of claim 10, wherein the supportive matrix comprises a tissue sealant or a fibrin sealant.

13. The method of claim 1, wherein an effective amount is an amount effective to increase osteoblast-tenocyte interface compared to osteoblast-tenocyte interface of a bone-tendon interface treated without PEP.

14. The method of claim 1, wherein an effective amount is an amount effective to improve at least one histological measure of the tendon-bone interface compared to a bone-tendon interface treated without PEP.

15. The method of claim 14, wherein the histological measure comprises an increase fiber continuity, an increase fiber parallel orientation, an increase collagen fiber density, a decrease vascularity, or a decrease cellularity compared to a bone-tendon interface treated without PEP.

16. The method of claim 1, wherein an effective amount is an amount effective to increase expression of at least one gene that promotes repair of a damaged tendon-bone interface.

17. The method of claim 16, wherein the gene encodes type I fibrillar collagen (Col1), type III fibrillar collagen (Col3), scleraxis BHLH transcription factor (SCX), tenomodulin (TNMD), decorin (DCN), or insulin-like growth factor 1 (IGF-1) in tissues of the tendon-bone interface.

18. The method of claim 1, wherein an effective amount is an amount effective to increase at least one biomechanical measure of the tendon-bone interface compared to a bone-tendon interface treated without PEP.

19. The method of claim 18, wherein the biomechanical measure comprises maximum load or stiffness.

20. The method of claim 1, wherein:
the damaged bone-tendon interface comprises complete separation of tendon from bone; and
the method further comprises surgically reattaching the tendon to the bone.

21. The method of claim 1, wherein:
the damaged tendon-bone interface comprises partial separation of tendon from bone; and
the method comprises implanting the PEP composition at a site effective for contacting the PEP composition with the damaged tendon-bone interface.
